(19) United States
(12) Patent Application Publication
(10) Pub. No.: US 2013/0150323 A1
(43) Pub. Date: Jun. 13, 2013

(54) USE OF ORAL HEPARIN PREPARATIONS TO TREAT URINARY TRACT DISEASES AND CONDITIONS

(71) Applicants: C. Lowell Parsons, Henderson, NV (US); Michael M. Goldberg, San Diego, CA (US)

(72) Inventors: C. Lowell Parsons, Henderson, NV (US); Michael M. Goldberg, San Diego, CA (US)

(73) Assignee: Urigen Pharmaceuticals, Inc, Wilmington, DE (US)

(21) Appl. No.: 13/577,363

(22) PCT Filed: Jan. 31, 2011
(86) PCT No.: PCT/US11/23108

§ 371 Date: Feb. 27, 2013

Related U.S. Application Data
(60) Provisional application No. 61/301,294, filed on Feb. 4, 2010.

(51) Int. Cl. A61K 31/727

(52) U.S. Cl. CPC A61K 31/727
USPC 514/56

(57) ABSTRACT

An improved method of treating lower urinary dysfunctional epithelium (LUDE) or a disease, condition, or syndrome associated with LUDE, including interstitial cystitis, comprises the step of administering orally a pharmaceutically effective quantity of heparin to a patient in need of treatment for LUDE or a disease, condition, or syndrome associated with LUDE in order to treat LUDE or a disease, condition, or syndrome associated with LUDE. The heparin can be administered together with a quantity of a penetration enhancer that is sufficient to result in a tissue concentration of heparin that is sufficient to treat LUDE or a disease, condition, or syndrome associated with LUDE. A suitable penetration enhancer is sodium N-[8-(2-hydroxybenzoyl)amino]caprylate. The method can further comprise the administration of at least one additional pharmaceutical composition to treat LUDE or a disease, condition, or syndrome associated with LUDE The invention further includes a pharmaceutical composition comprising: (1) a quantity of heparin that is pharmaceutically sufficient for the treatment of LUDE or a disease, condition, or syndrome associated with LUDE; and (b) at least one filler, excipient, or carrier; wherein the pharmaceutical composition is formulated for the treatment of LUDE or a disease, condition, or syndrome associated with LUDE.

### CROSS-REFERENCES TO RELATED APPLICATIONS

This PCT application claims priority from U.S. Provisional Application Ser. No. 61/301,294 by Parsons et al., entitled “Use of Oral Heparin Preparations to Treat Urinary Tract Diseases and Conditions,” and filed on Feb. 4, 2010, the contents of which are hereby incorporated herein in their entirety by this reference.

### FIELD OF THE INVENTION

This invention is directed to the use of oral heparin preparations for the treatment of diseases and conditions of the urinary tract, especially interstitial cystitis.

### BACKGROUND OF THE INVENTION

A large number of diseases and conditions occur in the lower urinary tract and are associated with one or more pelvic symptoms of pain, urge, frequency, or incontinence. In gynecologic patients, pelvic pain is referred to as chronic pelvic pain and may be of unknown origin or may be related to bacterial cystitis, fungal/yeast cystitis, vulvar vestibulitis, vulvodynia, dysparenunia, or endometriosis. Regardless of the perceived source of pelvic pain, in many cases the actual source of pain may be the bladder and/or the lower urinary tract. Frequency and urge together encompass the symptoms of overactive bladder. Overactive bladder may also be associated with incontinence, particularly urge incontinence.

In both male and female patients that are treated with cytotoxic therapies for cancer, this may result in any one or more lower urinary tract symptoms of pelvic pain, urge, frequency or incontinence. Localized radiation therapy to the pelvis which occurs due to bladder, cervical, ovarian, rectum, colon, vagina/vulva or prostate cancer therapy, may result in damaging the epithelium of the bladder wall leading to one or more of lower urinary tract symptoms of pain, urge, and/or frequency. Cytotoxic cancer chemotherapy, most notably cyclophosphamide and ifosfamide treatment for breast cancer patients (male and female) may also lead to the same series of symptoms.

In male patients, any one or more lower urinary tract pelvic symptoms of pelvic pain, urge, frequency or incontinence is observed in patients with prostatitis, chronic pelvic pain syndrome, urethral syndrome, or overactive bladder.

There are no specific treatments for lower urinary tract pelvic pain and instead patients are prescribed oral NSAIDS such as aspirin or acetaminophen. For severe chronic pain, some subjects rely on oral and/or transdermal narcotics which typically results in an irreversible worsening of symptoms.

For the symptoms of urinary urge and frequency, also termed overactive bladder, oral anticholinergic drugs such as detroloxybutynin chloride (Ditropan XL®) and tolterodine (Detrusitol®, Detrol LA®) reduce the contraction of the smooth muscle of the bladder wall. However, these drugs do not treat the underlying cause of the problem. Additionally, these drugs may result in side effects such as dry mouth, constipation, headache, blurred vision, hypertension, drowsiness, and urinary retention in approximately 50% of patients receiving them. The benefits of these drugs do not appear to overcome their risks/detriments since only 20% of patients refill their prescriptions.

There is one agent, Mesnex® (mesna) that is used for the prevention of hemorrhagic cystitis due to ifosfamide treatment in cancer patients. This agent is a detoxifying agent and binds and detoxifies the cancer drug. The drug does not treat acute pain and actually results in very high frequency of adverse events (all AEs for IV=85%, for oral=89%), most notable adverse events are nausea, vomiting, and constipation.

Although heparinoid-based therapy (heparin or the oral agent pentosan polysulfate sodium [PPS]) is an effective treatment for interstitial cystitis (IC), patients may require several months of therapy or more before they experience relief of pain and urgency/frequency (P. M. Hanno, “Analysis of Long-Term Elmiron Therapy for Interstitial Cystitis.” Urology 49(Suppl 5A): 93-99 (1997)). Heparinoids, which are believed to augment the dysfunctional epithelium that is present in many cases of the disease, take time to reach full effectiveness in reversing the disease process and thereby reducing symptoms. (C. L. Parsons, “Epithelial Coating Techniques in the Treatment of Interstitial Cystitis,” Urology 49(Suppl 5A): 100-104 (1997)). In addition, particularly in severe or long-standing cases of IC, there is significant upregulation of the sensory nerves in the bladder. (T J. Christmas et al., “Nerve Fibre Proliferation in Interstitial Cystitis.” Virchows Archiv. A Pathol. Anat. 416: 447-451 (1990); X. Pang et al., “Increased Number of Substance P Positive Nerve Fibres in Interstitial Cystitis,” Br. J. Urol. 75:744-750 (1995); C A. Buffington & S. A. Wolfe, Jr., “High Affinity Binding Sites for [³H]Substance P in Urinary Bladders of Cats with Interstitial Cystitis,” J. Urol. 160:605-611 (1998)). Heparinoids allow natural downregulation of the nerves over time by gradually restoring the barrier function of the mucus and thus preventing further irritation by urinary constituents such as potassium (J. C. Nickel et al., “Randomized, Double-Blind, Dose-Ranging Study of Pentosan Polysulfate Sodium (PPS) for Interstitial Cystitis (IC),” J. Urol. 165(5 Suppl): 67 (2001); C. L. Parsons et al., :Effect of Pentosan Polysulfate Therapy on Intravesical Potassium Sensitivity,” Urology 59: 329-333 (2002)). The use of heparinoids does not provide immediate symptom relief without destroying the nerve endings (T. W. Cannon & M. B. Chancellor, “Pharmacotherapy of the Overactive Bladder and Advances in Drug Delivery,” Clin. Obstet. Gynecol. 45: 205-17 (2002); M. B. Chancellor & N. Yoshimura, “Treatment of Interstitial Cystitis,” Urology 63(3 Suppl 1): 85-89 (2004); M. Lazzeri et al., “Intravesical Infusion of Resiniferatoxin by a Temporary in Situ Drug Delivery System to Treat Interstitial Cystitis: A Pilot Study,” Eur. Urol. 45: 98-102 (2004)) or employing narcotics.

Although heparinoids have proven effective for the treatment of IC and similar conditions, as described above, heparin itself has not been available for oral administration and has not been used to treat IC or similar conditions by oral administration to patients suffering from these diseases and conditions.

Intravesical agents have been used for many years as adjuncts to oral treatment regimens or as second-line therapies for IC. One of the most widely used is heparin, which is effective in approximately 50% of patients treated. Heparin is a sulfated polysaccharide that is believed to augment the protective effect of the natural bladder surface mucus. Intravesical heparinoid agents alone, however, do not produce immediate and sustained relief of IC symptoms. Like the oral heparinoids, they take several months to produce symptom relief. Also, as indicated above, heparin has not been available for oral administration.

Other treatments have also been tried, with limited success. For example, treatments with dimethylsulfoxide (DMSO), approved for IC in 1977 on the basis of data from uncontrolled trials, can be useful with weekly intravesical instillations for 6 to 8 weeks then every two weeks for 3-12 months for maintenance. However DMSO therapy results in benefit for approximately only 50% of IC patients treated and the treatment takes a long time to reduce symptoms. Furthermore, this therapy causes pain that is unrelieved by local anesthetics by themselves due to their lack of absorption into the bladder wall. Narcotics are given for immediate relief of symptoms; however, they are only minimally effective. The use of narcotics, of course, carries a significant risk of tolerance and addiction. Some patients benefit from a formal 8- to 12-week, one-on-one course of behavior modification. Patients are also advised to avoid potassium-rich foods, particularly citrus fruits, tomatoes, chocolate, and coffee.

Many urologists treat interstitial cystitis patients with their own “homebrew” of drugs by administering the drug(s) or mixtures thereof into the lumen of the bladder. As these procedures are typically done in the office without any quantitative assessment of severity of initial symptoms prior to or subsequent to treatment, there is no scientific rigor in assessing the benefit of these treatments. Consequently, patients are treated with drugs in their non-approved indications with no real scientific guidance as to whether the patient will benefit from the treatment or not.

Consequently, there is a tremendous need for scientifically-validated and improved treatments that provide immediate relief for treating lower urinary tract symptoms and disorders, particularly those with severe interstitial cystitis. Additionally, these treatments should be based on validated quantitative assessment of benefit, not on wishful thinking which has been the basis of urologists “homebrew” treatments that are not assessed quantitatively. There is a particular need for improved treatments and compositions for use in those treatments that provide immediate relief and do not require several months until the patients experience relief. In particular, there is a need for improved oral treatments for IC and other related conditions because oral treatments are far easier for the patient to administer and tolerate than intravesical treatments and do not carry the risk that invasive intravesical treatments may carry, such as the risk of injury or infection from catheterization.

Previously, Parsons (C. L. Parsons, “Evidence-based strategies for recognizing and managing IC,” Contemporary Urology, February 2003, pp. 22-35), published a recipe of three FDA-approved drug components for the treatment of interstitial cystitis, which is a painful bladder disorder of unknown etiology. The components were 80 mg lidocaine (8 ml of 1% lidocaine), 40,000 units heparin (4 ml of 10,000 units/ml heparin sodium), and 252 mg bicarbonate (3 ml of 8.4% sodium bicarbonate) in a total aqueous volume of 15 ml.

An additional limitation of the Parsons approach, as described in the 2003 Contemporary Urology article, is that components have to be measured out immediately before use from three separate solutions. In many treatment settings such as clinics or doctor's offices there are neither the pharmaceutical personnel resources qualified to measure out these components from stock solutions or the possibility exists of accidental mis-measurement leading to the potential for incorrect treatment or lidocaine overdose. Additionally, this mixing in a non-sterile environment may result in contamination with an infectious agent or other detrimental component that would be directly instilled in a compromised bladder.

The urinary bladder has a mucus layer that coats the outer apical membrane of the bladder surface epithelial cells. This mucus layer is in part composed of proteoglycans that contain glycosaminoglycans (such as heparan) and renders the epithelium impermeable to small solutes (2). When experimentally injured, however, the bladder epithelium of rodents and humans becomes abnormally permeable. Studies show that IC patients have defective bladder surface mucus that renders the bladder epithelium permeable to small molecules such as urea; normal asymptomatic controls, in contrast, have no such leak (2). The abnormally high permeability seen in IC patients results in increased absorption of urinary solutes into the bladder wall and provokes tissue reactions that cause symptoms. Potassium, present in urine in relatively high concentrations of 30 to 120 meq/L (4), is particularly toxic: upon absorption, it depolarizes bladder nerves and muscles and causes urgency, pain, incontinence, and tissue injury.

This potassium hypothesis explains all of the clinical observations seen in IC. Normal human volunteers will neither absorb nor experience symptoms after an intravesical challenge of potassium. However, after chemical injury of the mucus layer with protamine, these same normal volunteers upon placement of a 0.4 M potassium solution into the bladder will absorb it and complain of urgency and pain (5). This chemical injury was reversed by intravesically administered heparin (5). Moreover, it was shown that 80% of IC patients report these same symptoms in response to a potassium solution placed into the bladder. This basic physiologic process appears to be the primary cause for IC, overactive bladder (OAB), prostatitis (CP/CPPS), urethral syndrome (US) and gynecologic chronic pelvic pain (CPP) unifying them into one disease, lower urinary dysfunctional epithelium (LUDE) (2,11).

Based on the discovery that glycosaminoglycans (such as heparin) are important in protecting the bladder epithelium research was conducted to determine if administering heparinoids to interstitial cystitis patients could successfully treat this disease. Since no oral form of heparin has previously been available, a similar sulfated polysaccharide, pentosanpolysulfate (Elmiron) was tested in double blind studies and was found to significantly improve patients, with a good response in 34% of patients on active drug versus 16% on placebo (14). The availability of this drug orally is very limited, about 2-3%, and many patients not responding were felt in part due to the low levels that were obtained in urine. To successfully treat patients not responding to oral medication, a clinical trial was performed with heparin that was administered chronically with a catheter directly into the bladder and found it to be successfully at improving patients (13). However, as described above, intravesical administration into the urinary bladder via a catheter is uncomfortable for the patient and can present at least potential risks of injury and infection. Therefore, there is a need for compositions and methods that can be used to treat IC and other related diseases and conditions by the oral administration of heparin.

Heparinoids have been successfully reported to treat radiation cystitis (12). In addition, one could theorize that other disorders of the bladder epithelium, as noted above, could also benefit from heparinoid therapy such as overactive bladder, chemically induced cystitis (such as those caused by oncology drugs as occurs with cytoxan), gynecologic chronic pelvic pain (10), prostatitis and even urinary stone disease (11,15).

Since no oral form of heparin currently is available to treat the urologic diseases noted above, it was used intravesically to treat interstitial cystitis. Recently, experimental evidence suggests that oral forms of heparin can potentially be developed that may result in significant absorption and now open the possibility that oral heparin could be used to treat urologic diseases (16).

Therefore, there is an unfulfilled need to develop oral preparations containing heparin for the treatment of diseases and conditions such as interstitial cystitis and other urinary tract diseases and conditions, described generally as lower urinary dysfunctional epithelium (LUDE), which can include, in addition to IC, overactive bladder (OAB), prostatitis (CP/CPPS), urethral syndrome (US) and gynecologic chronic pelvic pain (CPP).

### SUMMARY OF THE INVENTION

Accordingly, we have developed methods for the use of oral heparin to treat interstitial cystitis and other urinary tract diseases and conditions, as well as compositions including oral heparin that are suitable for use in such methods, to meet the need for an effective oral therapy for interstitial cystitis and other urinary tract diseases and conditions employing heparin as a pharmacologically active agent.

One aspect of the invention is a method of treating lower urinary dysfunctional epithelium (LUDE) or a disease, condition, or syndrome associated with LUDE, including interstitial cystitis, comprising the step of administering orally a pharmaceutically effective quantity of heparin to a patient in need of treatment for LUDE.

In one alternative, the heparin has a molecular weight of from about 8,000 daltons to about 40,000 daltons. In another alternative, the heparin has a molecular weight of from about 2,000 daltons to about 8,000 daltons.

Typically, the heparin is administered as a salt with a positively charged counterion selected from the group consisting of sodium, trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, dibenzylpiperidine, N-benzyl-β-phenethylamine, dehydroabietylamine, N,N′-bisdehydroabietylamine, glucamine, N-methylglucamine, collidine, quinine, quinoline, lysine, and arginine. A preferable counterion is sodium.

Typically, the dosage of heparin is from about 25 units to about 25,000 units. More typically, the dosage of heparin is from about 100 units to about 15,000 units. Preferably, the dosage of heparin is from about 250 units to about 5,000 units. More preferably, the dosage of heparin is from about 500 units to about 2,500 units.

Typically, the heparin is administered orally at a frequency of from six times daily to once per week. Preferably, the heparin is administered orally four times daily, three times daily, twice daily, or once daily.

Typically, the heparin is administered together with a quantity of a penetration enhancer that is sufficient to result in a tissue concentration of heparin that is sufficient to treat LUDE or a disease, condition, or syndrome associated with LUDE.

In one alternative, the penetration enhancer is selected from a N-acylated α-amino acid or a salt or bioisostere thereof and a N-acylated non-α-amino acid or a salt or bioisostere thereof.

A preferred penetration enhancer is a penetration enhancer selected from the group consisting of a compound of Formula (VI) and salts, analogues, or bioisosteres thereof:

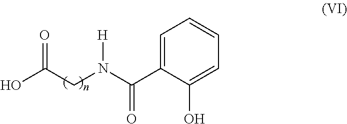

wherein n is 1, 2, 3, 4, 5, 6, 7, 8, 9, 10, or 11. A particularly preferred penetration enhancer is a compound or salt of Formula (VI) wherein n is 7, 8, or 9. A more particularly preferred penetration enhancer is sodium N-[8-(2-hydroxybenzoyl)amino]caprylate.

Typically, the heparin is administered orally in a dosage form selected from the group consisting of tablets, dragees, capsules, and solutions. Preferably, the heparin is administered orally in a solid dosage form selected from the group consisting of tablets, dragees, and capsules. More preferably, the heparin is administered orally in capsules; a particularly preferred form of capsule is a soft gelatin capsule.

In general, the method treats LUDE or a disease, condition, or syndrome associated with LUDE selected from the group consisting of interstitial cystitis, overactive bladder, prostatitis, urethral syndrome, and gynecological chronic pelvic pain. In one significant alternative, the method treats interstitial cystitis.

The method can further comprise the step of administering at least one additional pharmaceutical composition to treat LUDE or a disease, condition, or syndrome associated with LUDE. The at least one additional pharmaceutical composition can comprise a composition selected from the group consisting of: sodium pentosanpolysulfate; a composition comprising a heparinoid, a local anesthetic, and a buffering compound; an oral anticholinergic drug; mesna; dimethyl sulfoxide; an analgesic; and a narcotic.

Another aspect of the present invention is a pharmaceutical composition comprising:

(1) a quantity of heparin that is pharmaceutically sufficient for the treatment of LUDE or a disease, condition, or syndrome associated with LUDE; and

(2) at least one filler, excipient, or carrier;

wherein the pharmaceutical composition is formulated for the treatment of LUDE or a disease, condition, or syndrome associated with LUDE.

The pharmaceutical composition can further comprise a penetration enhancer as described above.

The pharmaceutical composition can be in a dosage form selected from the group consisting of tablets, dragees, capsules, and solutions. Preferably, the pharmaceutical composition is in a solid dosage form selected from the group consisting of tablets, dragees, and capsules. More preferably, the pharmaceutical composition is in capsule form, such as soft gelatin capsules.

### DETAILED DESCRIPTION OF THE INVENTION

An improved method of treatment of LUDE or a disease, condition, or syndrome associated with LUDE, including interstitial cystitis, employs the oral administration of heparin.

In general, a method according to the present invention comprises the step of administering orally a pharmaceutically effective quantity of heparin to a patient in need of treatment for LUDE or a disease, condition, or syndrome associated with LUDE in order to treat LUDE or a disease, condition, or syndrome associated with LUDE.

Diseases, conditions, or syndromes associated with LUDE include, but are not limited to, interstitial cystitis (IC), overactive bladder (OAB), prostatitis (CP/CPPS), urethral syndrome (US) and gynecologic chronic pelvic pain (CPP).

Heparin exists in a variety of forms characterized by different degrees of sulfation. Typically, heparin has a molecular weight of from about 2 kDa to about 40 kDa. Heparin is characterized by repeating units of disaccharides containing a uronic acid (glucuronic acid or iduronic acid) and glucosamine, which is either N-sulfated or N-acetylated. The sugar residues can be further O-sulfated at the C-6 and C-3 positions and the C-2 position of the uronic acid. There are at least 32 potential unique disaccharide units in this class of compounds, to which heparin belongs. Five examples of sugars occurring in heparin are: (1) α-L-iduronic acid 2-sulfate; (2) 2-deoxy-2-sulfamino-α-D-glucose 6-sulfate; (3) β-D-glucuronic acid, (4) 2-acetamido-2-deoxy-α-D-glucose, and (5) α-L-iduronic acid. Heparin is measured by its specific anticoagulation activity in units. As used herein, the term “units” refers to specific activity in International Units (IU) and/or United States Pharmacopoeia (USP) units. As used herein, the term “USP unit” refers to the quantity of heparin that prevents 1.0 ml of citrated sheep plasma from clotting for 1 hour after the addition of 0.2 ml of 1% CaCl₂ at 20° C. when compared to a USP reference standard (defined as units/ml). As used herein, the term “International Unit” or “IU” refers to the quantity of heparin that is active in assays as established by the Fifth International standard for Unfractionated Heparin (WHO-5) (defined as International Units/ml) (R. J. Linhardt & N. S. Gunay, Semin. Thromb. Hemost. 25: 5-16 (1999)). In some embodiments, heparin is a higher molecular weight species ranging from 8,000 to 40,000 daltons. As used herein, the phrase “low-molecular-weight heparins” refers to a lower molecular weight (LMW) species ranging from 2,000 to 8,000 daltons. LMW heparins are made by enzymatic or chemical controlled hydrolysis of unfractionated heparin and have very similar chemical structure to unfractionated heparin except for some changes that may have been introduced due to the enzymatic or chemical treatment. While not intending to limit the mechanism of action of the invention's compositions, it is the inventor's view that mechanism of action of these drugs is similar to that of full-length heparin. LMW heparins are usually isolated from bulk heparin.

Because of the negative charge of these polysaccharides due to the occurrence of sulfate groups and/or carboxylic acid groups in them, they are administered in the form of salts, with an appropriate cation to neutralize the negative charges on the acid groups. Typically, the cation is sodium. However, other physiologically tolerable counterions that do not induce urinary tract dysfunctions such as magnesium and aluminum, as well as salts made from physiologically acceptable organic bases such as, but not limited to, trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, dibenzylpiperidine, N-benzyl-β-phenethylamine, dehydroabietylamine, N,N′-bisdehydroabietylamine, glucamine, N-methylglucamine, collidine, quinine, quinoline, and basic amino acids such as lysine and arginine, can be used. These salts may be prepared by methods known to those skilled in the art. However, it is generally undesirable to use potassium as a counterion due to its role in the etiology of the conditions and syndromes being treated.

Typically, the dose of heparin that is administered is from about 25 units to about 25,000 units, administered orally. More typically, the dose of heparin that is administered orally is from about 100 units to about 15,000 units. Preferably, the dose of heparin that is administered is from about 250 units to about 5,000 units. More preferably, the dose of heparin that is administered is from about 500 units to about 2,500 units.

Typically, in methods according to the present invention, the frequency of oral administration of heparin is from six times daily to once per week. For example, but not by way of limitation, the heparin can be administered six times daily, five times daily, four times daily, three times daily, twice daily, once daily, once every two days, once every three days, or once a week. Preferably, the heparin is administered four times daily, three times daily, twice daily, or once daily.

Typically, in methods according to the present invention, the heparin is administered together with a quantity of a penetration enhancer that is sufficient to result in a tissue concentration of heparin that is sufficient to treat LUDE or a disease, condition, or syndrome associated with LUDE. A number of penetration enhancers are known in the art. For example, the penetration enhancer can be an N-acylated α-amino acid or a salt or bioisostere thereof, or a N-acylated non-α-amino acid or a salt or bioisostere thereof (A. Leone-Bay et al., “N-Acylated α-Amino Acids as Novel Oral Delivery Agents for Proteins,” J. Med. Chem. 38: 4263-4269 (1995); A. Leone-Bay et al., “4-[4-(2 Hydroxybenzoyl)amino] Phenyl Butyric Acid as a Novel Delivery Agent for Recombinant Human Growth Hormone,” J. Med. Chem. 39: 2751-2576 (1996); A. Leone-Bay et al., “Oral Delivery of Heparin” in Controlled Drug Delivery (ACS Symposium Series, vol. 752, 2000), ch. 6, pp. 54-64; U.S. Pat. No. 6,462,643 to Milstein et al.).

Penetration enhancers suitable for use in methods according to the present invention can include, but are not limited to the enhancers described below as (1)-(6).

One group of penetration enhancers is: (1) N-benzoyl-α-amino acids of Formula (I) and salts, analogues, or bioisosteres thereof:

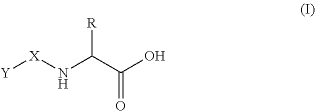

wherein the α-amino acid is selected from the group consisting of glycine, alanine, valine, leucine, phenylalanine, tyrosine, aspartic acid, glutamic acid, lysine, ornithine, arginine, and serine, wherein X is selected from the group consisting of C(O) and SO₂, and wherein Y is selected from the group consisting of phenyl and cyclohexyl.

Another group of penetration enhancers is: (2) derivatized leucines of Formula (II) and salts, analogues, or bioisosteres thereof:

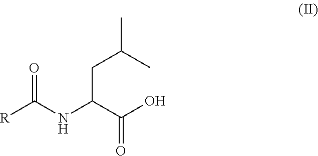

wherein R is selected from the group consisting of cyclohexyl, 2-methylcyclohexyl, 3-methylcyclohexyl, 4-methylcyclohexyl, cycloheptyl, cyclopentyl, cyclopropyl, 2-carboxycyclohexyl, benzoyl, 3-methoxyphenyl, 2-nitrophenyl, 3-nitrophenyl, 4-nitrophenyl, and (CH₂)₂cyclohexyl.

Yet another group of penetration enhancers is: (3) N-cyclohexanoylamino acids of Formula (III) and salts, analogues, or bioisosteres thereof:

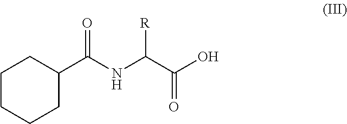

wherein R is selected from the group consisting of CH₂Ph, (CH₂)₃NHC(NH)NH₂, i-butyl, s-butyl, (CH₂)₄NH, CH₂(4-C₆H₄OH), (CH₂)₃NHC(O)NH₂, CH₂(imidazole), and phenyl.

Yet another group of penetration enhancers is: (4) derivatized phenylglycines of Formula (IV) and salts, analogues, or bioisosteres thereof:

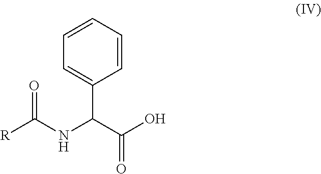

wherein R is selected from the group consisting of cyclohexyl, cyclopentyl, cycloheptyl, methylcyclohexyl, (CH₂)₂cyclohexyl, phenyl, and 2-hydroxyphenyl.

Still another group of penetration enhancers is (5): derivatives of 4-aminobenzoic acid, 2-(4-aminophenyl)acetic acid, 3-(4-aminophenyl)propionic acid, or 4-(4-aminophenyl)butyric acid of Formula (V) and salts, analogues, or bioisosteres thereof:

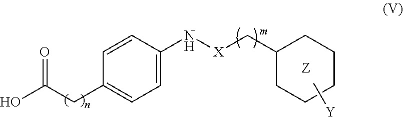

wherein: (a) Y is selected from the group consisting of H, F, 2-OH, 2,3-Ph, 4-Ph, 3,4-Ph, 4-OCH₃, 4-F, 2-Cl, 2-F, 2,4-(OH)₂, 3-CF₃, 3-Cl, 2-CH₃, 2,6-(OH)₂, 3-N(CH₃), 3,4-OCH₂O, 2,6-diCH₃, 2-COOH, 2-NO₂, 2-OCH₃, 3-NO₂, 2-OCF₃, 4-CH₃, and 4-i-Bu; (b) n is 0, 1, 2, 3, 4, or a vinyl group; (c) m is 0, 1, or 2, a vinyl group, a CHMe group, a CHEt group; a (CH₂)₂O group, a (CH₂)₂C═O group, or a (CH₂OH)₂ group; (d) X is C═O, SO₂, or CH₂; and (e) Z is phenyl, cyclohexyl, or cycloheptyl.

Yet another group of penetration enhancers is: (6) compounds of Formula (VI):

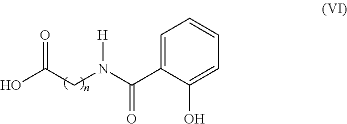

wherein n is 1, 2, 3, 4, 5, 6, 7, 8, 9, 10, or 11, and salts, analogues, or bioisosteres thereof. Preferred enhancers are compounds or salts of Formula (VI) that have n as 7, 8, or 9.

A particularly preferred penetration enhancer is sodium N-[8-(2-hydroxybenzoyl)amino]caprylate, also known as salcaprozate sodium or SNAC (S. A. Mousa et al., “Pharmacokinetics and Pharmacodynamics of Oral Heparin Solid Dosage Form in Healthy Human Subjects,” J. Clin. Pharmacol. 47: 1508-1520 (2007). This is the sodium salt of a compound of Formula (VI) with n equal to 7.

Other penetration enhancers are known in the art.

The exact formulation, route of administration and dosage can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps the dose frequency, will also vary according to the age, body weight, and response of the individual patient, as well as factors such as pharmacokinetic factors such as liver and kidney function.

In methods according to the present invention, the heparin is typically administered in a dosage form that is palatable and acceptable to the patient when administered orally. Many suitable dosage forms are known in the art.

For example, in general, dosage forms for the oral administration of heparin can be in the form of tablets, dragees, capsules, or solutions, although, for reasons of palatability and acceptability, solid dosage forms are typically preferred. Suitable solid dosage forms for the oral administration of heparin can be prepared by combining the heparin, the cationic counterion, the penetration enhancer, if present, and any other ingredients such as stabilizers, preservatives, or excipients, adding other inert ingredients as necessary to provide the correct volume of the mixture, and grinding the mixture to homogeneity. The resulting mixture can be pressed into tablets or dragees or incorporated into capsules, as described further below.

Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

In one particularly preferred dosage form for administration, heparin plus the penetration enhancer SNAC are incorporated into soft gelatin capsules.

Other suitable dosage forms are known in the art.

As used herein, terms such as “treating,” “treatment,” and analogous terminology does not imply a complete cure of LUDE or a disease, condition, or syndrome associated with LUDE, such as interstitial cystitis. The terms “treating,” “treatment,” or analogous terminology as used herein mean that a patient that is treated by a method according to the present invention achieves a detectable result of improvement with respect to LUDE or a disease, condition, or syndrome associated with LUDE, such as interstitial cystitis. Such a detectable result of improvement can be, but is not limited to, a reduction in pain, a reduction in urinary frequency, a reduction of urinary urgency, a reduction of incontinence, an increase in bladder capacity, a reduction of potassium permeability of the bladder epithelium, or any other objective or subjective result experienced by the patient.

Methods according to the present invention can be combined with other methods for treatment of LUDE or a disease, condition, or syndrome associated with LUDE, including interstitial cystitis. The pharmaceutical compositions used in these methods can be administered by oral or other routes as appropriate. Such methods include the administration of sodium pentosanpolysulfate, as described in U.S. Pat. No. 5,180,715 to Parsons, the intravesical administration of a composition comprising a heparinoid, a local anesthetic, and a buffering compound, as described in U.S. Pat. No. 7,414,039 to Parsons, and other methods described in PCT Patent Application Publication No. WO 2007/073397 by Flashner et al., such as the administration of oral anticholinergic drugs such as detroloxybutynin chloride (Ditropan XL®) or tolterodine (Detrusitol® or Detrol LA®), the administration of mesna (Mesnex®), the administration of dimethyl sulfoxide (DMSO), or the administration of analgesics or narcotics to control pain.

Another aspect of the present invention is a pharmaceutical composition comprising heparin in a form suitable for oral administration. The pharmaceutical composition is formulated for the treatment of LUDE or a disease, condition, or syndrome associated with LUDE, including interstitial cystitis.

In general, a pharmaceutical composition according to the present invention comprises:

(1) a quantity of heparin that is pharmaceutically sufficient for the treatment of LUDE or a disease, condition, or syndrome associated with LUDE; and

(2) at least one filler, excipient, or carrier;

wherein the pharmaceutical composition is formulated for the treatment of LUDE or a disease, condition, or syndrome associated with LUDE.

Typically, a pharmaceutical composition according to the present invention comprises a quantity of heparin from about 25 units to about 25,000 units. More typically, a pharmaceutical composition according to the present invention comprises a quantity of heparin from about 100 units to about 15,000 units. Preferably, a pharmaceutical composition according to the present invention comprises a quantity of heparin from about 250 units to about 5,000 units. More preferably, a pharmaceutical composition according to the present invention comprises a quantity of heparin from about 500 units to about 2,500 units.

Preferably, a pharmaceutical composition according to the present invention further comprises a penetration enhancer as described above. As indicated above, a particularly preferred penetration enhancer as used in a pharmaceutical composition according to the present invention is sodium N-[8-(2-hydroxybenzoyl)amino]caprylate (SNAG), as described above.

The dosage form of a pharmaceutical composition according to the present invention is typically a tablet, a dragee, a capsule, or a solution; preferably, the dosage form is a tablet, a dragee, or a capsule. A particularly preferred dosage form is a capsule, such as a soft gelatin capsule.

The following references may be useful in understanding the invention. These references are not necessarily prior art and are not identified herein as prior art. These references are referred to herein by numerals in parenthesis, e.g., (1).
1. Parsons, C. L., et al., Abnormal sensitivity to intravesical potassium in interstitial cystitis and radiation cystitis. Neurourol Urodyn. 1994; 13(5):515-20.
2. Parsons, C. L., The role of the urinary epithelium in the pathogenesis of interstitial cystitis/prostatitis/urethritis. Urology. 2007; 69(4 Suppl):9-16.
3. Hanno P M, Landis J R, Matthews-Cook Y, Kusek J, Nyberg L Jr The diagnosis of interstitial cystitis revisited: lessons learned from the National Institutes of Health Interstitial Cystitis Database study. J Urol. 1999 February; 161(2):553-7.
4. Parsons, C. L., et al., Abnormal urinary potassium metabolism in patients with interstitial cystitis. J Urol. 2005; 173(4):1182-5.
5. Parsons, C. L., et al., The role of urinary potassium in the pathogenesis and diagnosis of interstitial cystitis. J Urol. 1998; 159(6):1862-6; discussion 1866-7.
6. Hassan A A, Elgamal S A, Sabaa M A, Salem K Evaluation of intravesical potassium sensitivity test and bladder biopsy in patients with chronic prostatitis/chronic pelvic pain syndrome. Int J Urol. 2007 August; 14(8):738-42.
7. Daha, L., et al., Comparative (saline vs. 0.2M potassium chloride) assessment of maximum bladder capacity: a well tolerated alternative to the 0.4M potassium sensitivity test (PST). J Urol. 2001; 165(suppl):68.
8. Philip, J., S. Willmott, and P. Irwin, Interstitial cystitis versus detrusor overactivity: a comparative, randomized, controlled study of cystometry using saline and 0.3 M potassium chloride. J Urol. 2006; 175(2):566-70; discussion 570-1.
9. Abrams P, Hanno P, Wein A Overactive Bladder and Painful Bladder Syndrome: There Need not be Confusion. Neurourology and Urodynamics 24:149-150 (2005)
10. Parsons, C. L., et al., The prevalence of interstitial cystitis in gynecologic patients with pelvic pain, as detected by intravesical potassium sensitivity. Am J Obstet Gynecol. 2002; 187(5):1395-400.
11. Parsons C L, The potassium sensitivity test: a new gold standard for diagnosing and understanding the pathophysiology of interstitial cystitis. J Urol. 2009 August; 182(2):432-4
12. Parsons C L, Successful management of radiation cystitis with sodium pentosanpolysulfate. J Urol. 1986 October; 136(4):813-4
13. Parsons C L, et al Treatment of interstitial cystitis with intravesical heparin Br J Urol. 1994 May; 73(5):504-7).
14. Parsons C L et al, A quantitatively controlled method to study prospectively interstitial cystitis and demonstrate the efficacy of pentosanpolysulfate. J Urol. 1993 September; 150(3):845-8)
15. Parsons C L et al, Inhibition of sodium urate crystal adherence to bladder surface by polysaccharide. J Urol. 1985 September; 134(3):614-6.
16. Mousa S A et al, Pharmacokinetics and pharmacodynamics of oral heparin solid dosage form in healthy human subjects. J Clin Pharmacol. 2007 December; 47(12):1508-20.

### ADVANTAGES OF THE INVENTION

The present invention provides improved treatment methods and compositions for the oral treatment of LUDE, or a disease, condition, or syndrome associated with LUDE, including interstitial cystitis. The treatment methods and compositions according to the present invention provide improved oral delivery and bioavailability of the pharmacologically active agent heparin and are well accepted by patients. They are free of side effects and can be used together with other therapies for treatment of LUDE.

Methods according to the present invention possess industrial applicability for the preparation of a medicament for the oral treatment of LUDE, or a disease, condition, or syndrome associated with LUDE, including interstitial cystitis.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, and literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

1. A method of treating lower urinary dysfunctional epithelium (LUDE) or a disease, condition, or syndrome associated with LUDE comprising the step of administering orally a pharmaceutically effective quantity of heparin to a patient in need of treatment for LUDE or a disease, condition, or syndrome associated with LUDE in order to treat LUDE or a disease, condition, or syndrome associated with LUDE.

2. The method of claim 1 wherein the heparin has a molecular weight of from about 8,000 daltons to about 40,000 daltons.

3. The method of claim 1 wherein the heparin has a molecular weight of from about 2,000 daltons to about 8,000 daltons.

4. The method of claim 1 wherein the heparin is administered as a salt with a positively charged counterion selected from the group consisting of sodium, trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, dibenzylpiperidine, N-benzyl-β-phenethylamine, dehydroabietylamine, N,N′-bisdehydroabietylamine, glucamine, N-methylglucamine, collidine, quinine, quinoline, lysine, and arginine.

5. The method of claim 4 wherein the counterion is sodium.

6. The method of claim 1 wherein the dosage of heparin is from about 25 units to about 25,000 units.

7. The method of claim 6 wherein the dosage of heparin is from about 100 units to about 15,000 units.

8. (canceled)

9. (canceled)

10. The method of claim 1 wherein the heparin is administered orally at a frequency of from six times daily to once per week.

11. The method of claim 10 wherein the heparin is administered orally four times daily, three times daily, twice daily, or once daily.

12. The method of claim 1 wherein the heparin is administered together with a quantity of a penetration enhancer that is sufficient to result in a tissue concentration of heparin that is sufficient to treat LUDE or a disease, condition, or syndrome associated with LUDE.

13. The method of claim 12 wherein the penetration enhancer is selected from a N-acylated α-amino acid or a salt or bioisostere thereof and a N-acylated non-α-amino acid or a salt or bioisostere thereof.

14. The method of claim 12 wherein the penetration enhancer is selected from the group consisting of a N-benzoyl-α-amino acid of Formula (I) and salts, analogues, or bioisosteres thereof: 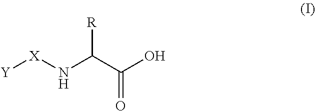
wherein the α-amino acid is selected from the group consisting of glycine, alanine, valine, leucine, phenylalanine, tyrosine, aspartic acid, glutamic acid, lysine, ornithine, arginine, and serine, wherein X is selected from the group consisting of C(O) and SO₂, and wherein Y is selected from the group consisting of phenyl and cyclohexyl.

15. The method of claim 12 wherein the penetration enhancer is selected from the group consisting of a derivatized leucine of Formula (II), and salts, analogues, or bioisosteres thereof: 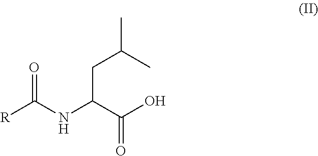
wherein R is selected from the group consisting of cyclohexyl, 2-methylcyclohexyl, 3-methylcyclohexyl, 4-methylcyclohexyl, cycloheptyl, cyclopentyl, cyclopropyl, 2-carboxycyclohexyl, benzoyl, 3-methoxyphenyl, 2-nitrophenyl, 3-nitrophenyl, 4-nitrophenyl, and (CH₂)₂cyclohexyl.

16. The method of claim 12 wherein the penetration enhancer is selected from the group consisting of a N-cyclohexanoylamino acid of Formula (III) and salts, analogues, or bioisosteres thereof: 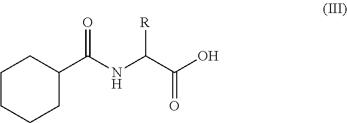
wherein R is selected from the group consisting of CH₂Ph, (CH₂)₃NHC(NH)NH₂, s-butyl, (CH₂)₄NH, CH₂(4-C₆H₄OH), (CH₂)₃NHC(O)NH₂, CH₂(imidazole), and phenyl.

17. The method of claim 12 wherein the penetration enhancer is selected from the group consisting of a derivatized phenylglycine of Formula (IV) and salts, analogues, or bioisosteres thereof: 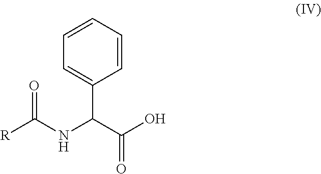
wherein R is selected from the group consisting of cyclohexyl, cyclopentyl, cycloheptyl, methylcyclohexyl, (CH₂)₂cyclohexyl, phenyl, and 2-hydroxyphenyl.

18. The method of claim 12 wherein the penetration enhancer is selected from the group consisting of 4-aminobenzoic acid, 2-(4-aminophenyl)acetic acid, 3-(4-aminophenyl)propionic acid, or 4-(4-aminophenyl)butyric acid of Formula (V) and salts, analogues, or bioisosteres thereof: 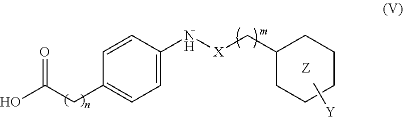
wherein: (a) Y is selected from the group consisting of H, F, 2-OH, 2,3-Ph, 4-Ph, 3,4-Ph, 4-OCH₃, 4-F, 2-Cl, 2-F, 2,4-(OH)₂, 3-CF₃, 3-Cl, 2-CH₃, 2,6-(OH)₂, 3-N(CH₃), 3,4-OCH₂O, 2,6-diCH₃, 2-COOH, 2-NO₂, 2-OCH₃, 3-NO₂, 2-OCF₃, 4-CH₃, and 4-i-Bu; (b) n is 0, 1, 2, 3, 4, or a vinyl group; (c) m is 0, 1, or 2, a vinyl group, a CHMe group, a CHEt group; a (CH₂)₂O group, a (CH₂)₂C═O group, or a (CH₂OH)₂ group; (d) X is C═O, SO₂, or CH₂; and (e) Z is phenyl, cyclohexyl, or cycloheptyl.

19. The method of claim 12 wherein the penetration enhancer is selected from the group consisting of a compound of Formula (VI) and salts, analogues, or bioisosteres thereof: 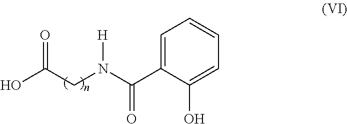
wherein n is 1, 2, 3, 4, 5, 6, 7, 8, 9, 10, or 11.

20. The method of claim 19 wherein the penetration enhancer is a compound or salt of Formula (VI) wherein n is 7, 8, or 9.

21. The method of claim 20 wherein the penetration enhancer is sodium N-[8-(2-hydroxybenzoyl)amino]caprylate.

22. The method of claim 1 wherein the heparin is administered orally in a dosage form selected from the group consisting of tablets, dragees, capsules, and solutions.

23. (canceled)

24. The method of claim 22 wherein the heparin is administered orally in capsules.

25. The method of claim 24 wherein the heparin is administered in soft gelatin capsules.

26. The method of claim 25 wherein the heparin is administered together with sodium N-[8-(2-hydroxybenzoyl)amino]caprylate.

27. The method of claim 1 wherein the method treats LUDE or a disease, condition, or syndrome associated with LUDE selected from the group consisting of interstitial cystitis, overactive bladder, prostatitis, urethral syndrome, and gynecological chronic pelvic pain.

28. The method of claim 27 wherein the method treats interstitial cystitis.

29. The method of claim 1 wherein the method further comprises the step of administering at least one additional pharmaceutical composition to treat LUDE or a disease, condition, or syndrome associated with LUDE.

30. The method of claim 29 wherein the at least one pharmaceutical composition comprises a composition selected from the group consisting of: sodium pentosanpolysulfate; a composition comprising a heparinoid, a local anesthetic, and a buffering compound; an oral anticholinergic drug; mesna; dimethyl sulfoxide; an analgesic; and a narcotic.

31. The method of claim 29 wherein the method treats interstitial cystitis.

32. A pharmaceutical composition comprising:
(a) a quantity of heparin that is pharmaceutically sufficient for the treatment of LUDE or a disease, condition, or syndrome associated with LUDE; and
(b) at least one filler, excipient, or carrier;
wherein the pharmaceutical composition is formulated for the treatment of LUDE or a disease, condition, or syndrome associated with LUDE.

33. The pharmaceutical composition of claim 32 wherein the pharmaceutical composition comprises a quantity of heparin from about 25 units to about 25,000 units.

34. The pharmaceutical composition of claim 33 wherein the pharmaceutical composition comprises a quantity of heparin from about 100 units to about 15,000 units.

35. The pharmaceutical composition of claim 34 wherein the pharmaceutical composition comprises a quantity of heparin from about 250 units to about 5,000 units.

36. (canceled)

37. The pharmaceutical composition of claim 32 wherein the composition further comprises a quantity of a penetration enhancer that is sufficient to result in a tissue concentration of heparin that is sufficient to treat LUDE or a disease, condition, or syndrome associated with LUDE.

38. The pharmaceutical composition of claim 37 wherein the penetration enhancer is selected from a N-acylated α-amino acid or a salt or bioisostere thereof and a N-acylated non-α-amino acid or a salt or bioisostere thereof.

39. The pharmaceutical composition of claim 37 wherein the penetration enhancer is selected from the group consisting of a N-benzoyl-α-amino acid of Formula (I) and salts, analogues, or bioisosteres thereof: 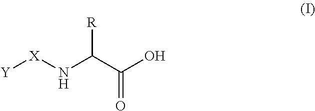
wherein the α-amino acid is selected from the group consisting of glycine, alanine, valine, leucine, phenylalanine, tyrosine, aspartic acid, glutamic acid, lysine, ornithine, arginine, and serine, wherein X is selected from the group consisting of C(O) and SO₂, and wherein Y is selected from the group consisting of phenyl and cyclohexyl.

40. The pharmaceutical composition of claim 37 wherein the penetration enhancer is selected from the group consisting of a derivatized leucine of Formula (II), and salts, analogues, or bioisosteres thereof: 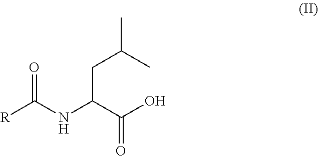
wherein R is selected from the group consisting of cyclohexyl, 2-methylcyclohexyl, 3-methylcyclohexyl, 4-methylcyclohexyl, cycloheptyl, cyclopentyl, cyclopropyl, 2-carboxycyclohexyl, benzoyl, 3-methoxyphenyl, 2-nitrophenyl, 3-nitrophenyl, 4-nitrophenyl, and (CH₂)₂cyclohexyl.

41. The pharmaceutical composition of claim 37 wherein the penetration enhancer is selected from the group consisting of a N-cyclohexanoylamino acid of Formula (III) and salts, analogues, or bioisosteres thereof: 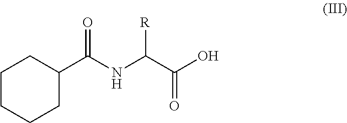
wherein R is selected from the group consisting of CH₂Ph, (CH₂)₃NHC(NH)NH₂, i-butyl, s-butyl, (CH₂)₄NH, CH₂(4-C₆H₄OH), (CH₂)₃NHC(O)NH₂, CH₂(imidazole), and phenyl.

42. The pharmaceutical composition of claim 37 wherein the penetration enhancer is selected from the group consisting of a derivatized phenylglycine of Formula (IV) (IV) and salts, analogues, or bioisosteres thereof: 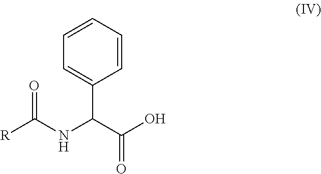
wherein R is selected from the group consisting of cyclohexyl, cyclopentyl, cycloheptyl, methylcyclohexyl, (CH₂)₂cyclohexyl, phenyl, and 2-hydroxyphenyl.

43. The pharmaceutical composition of claim 37 wherein the penetration enhancer is selected from the group consisting of 4-aminobenzoic acid, 2-(4-aminophenyl)acetic acid, 3-(4-aminophenyl)propionic acid, or 4-(4-aminophenyl)butyric acid of Formula (V) and salts, analogues, or bioisosteres thereof: 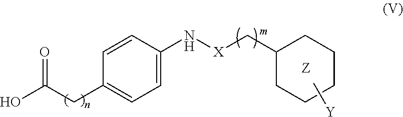
wherein: (a) Y is selected from the group consisting of H, F, 2-OH, 2,3-Ph, 4-Ph, 3,4-Ph, 4-OCH₃, 4-F, 2-Cl, 2-F, 2,4-(OH)₂, 3-CF₃, 3-Cl, 2-CH₃, 2,6-(OH)₂, 3-N(CH₃), 3,4-OCH₂O, 2,6-diCH₃, 2-COOH, 2-NO₂, 2-OCH₃, 3-NO₂, 2-OCF₃, 4-CH₃, and 4-i-Bu; (b) n is 0, 1, 2, 3, 4, or a vinyl group; (c) m is 0, 1, or 2, a vinyl group, a CHMe group, a CHEt group; a (CH₂)₂O group, a (CH₂)₂C═O group, or a (CH₂OH)₂ group; (d) X is C═O, SO₂, or CH₂; and (e) Z is phenyl, cyclohexyl, or cycloheptyl.

44. The pharmaceutical composition of claim 37 wherein the penetration enhancer is selected from the group consisting of a compound of Formula (VI) and salts, analogues, or bioisosteres thereof: 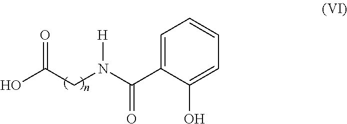
wherein n is 1, 2, 3, 4, 5, 6, 7, 8, 9, 10, or 11.

45. The pharmaceutical composition of claim 44 wherein the penetration enhancer is a compound or salt of Formula (VI) wherein n is 7, 8, or 9.

46. The pharmaceutical composition of claim 45 wherein the penetration enhancer is sodium N-[8-(2-hydroxybenzoyl)amino]caprylate.

47. The pharmaceutical composition of claim 32 wherein the pharmaceutical composition is in a dosage form selected from the group consisting of tablets, dragees, capsules, and solutions.

48. (canceled)

49. The pharmaceutical composition of claim 47 wherein the pharmaceutical composition is in capsule form.

50. The pharmaceutical composition of claim 49 wherein the pharmaceutical composition is in the form of soft gelatin capsules.

51. The pharmaceutical composition of claim 50 wherein the pharmaceutical composition further comprises sodium N-[8-(2-hydroxybenzoyl)amino]caprylate.

52. The pharmaceutical composition of claim 34 wherein the pharmaceutical composition is formulated for the treatment of LUDE or a disease, condition, or syndrome associated with LUDE selected from the group consisting of interstitial cystitis, overactive bladder, prostatitis, urethral syndrome, and gynecological chronic pelvic pain.

53. The pharmaceutical composition of claim 52 wherein the pharmaceutical composition is formulated for the treatment of interstitial cystitis.
